(19) United States
(12) Patent Application Publication
(10) Pub. No.: US 2004/0037880 A1
(43) Pub. Date: Feb. 26, 2004

(54) Extended release formulation of divalproex sodium

(76) Inventors: Pratik Kumar, Muzaffarpur (IN); Girish Kumar Jain, Pitampura (IN); Ashok Rampal, Amritsar (IN)

Correspondence Address: Jayadeep R. Deshmukh, Esq., Ranbaxy Pharmaceuticals Inc., Suite 2100, 600 College Road East, Princeton, NJ, 08540, US

(21) Appl. No.: 10/456,008

(22) Filed: Jun. 6, 2003

(30) Foreign Application Priority Data
Jun. 7, 2002 (IN) 615/DEL/2002

(51) Int. Cl. A61K 9/22; A61K 31/19

(52) U.S. Cl. 424/468; 514/557

(57) ABSTRACT

The present invention relates to an extended release pharmaceutical composition comprising valproic acid, a pharmaceutically acceptable salt, ester, or amide thereof or divalproex sodium.

### FIELD OF THE INVENTION

The present invention relates to an extended release pharmaceutical composition comprising valproic acid, a pharmaceutically acceptable salt, ester, or amide thereof or divalproex sodium.

### BACKGROUND OF THE INVENTION

Valproic acid, valpromide, and pharmaceutically acceptable salts and esters of the acid are effectively used in the treatment of mania, migraine and epilepsy. After ingestion, they dissociate to the valproate ion within the gastrointestinal tract, which on absorption produces the desired therapeutic effect.

Valproic acid and its derivatives are either liquid or liquefy rapidly and become sticky. Further, most of them are extremely hygroscopic in nature. These physicochemical properties pose serious problems during manufacture of pharmaceutical compositions.

Valproic acid and its derivatives also suffer from another disadvantage of relatively short elimination half-life. For example, a short half-life of between 6-17 hours in adults and 4-14 hours in children has been reported for valproic acid. Frequent dosing is thus necessary to maintain reasonably stable plasma concentrations. However, it results in inconvenience to the patient, leading to reduced patient compliance. Moreover, widely fluctuating plasma concentrations of the drug also result in administration of erratic amounts of the drug.

To overcome the disadvantages, a number of research endeavors have been directed towards preparing controlled release formulations that permits once a day dosing and thereby helps in maintaining a reasonably stable plasma concentration.

For example, U.S. Pat. No. 6,419,953 discloses a controlled release tablet dosage form containing a valproate compound. The controlled release tablet dosage form is described as a hydrophilic matrix including a mixture of a valproate compound, hydroxypropyl methylcellulose, lactose, microcrystalline cellulose, and silicon dioxide having an average particle size ranging between about 1 micron and about 10 microns. The patent further teaches that the addition of either 1% silicon dioxide or/and 5% microcrystalline cellulose to the hydrophilic matrix formulations of the invention increases tablet hardness. However the problem of sticking still persists when conventionally used grades of silicon dioxide are employed, and can be overcome only by the use of a special grade silicon dioxide (Syloid® 244) having a larger average particle size ranging from about 1 micron to about 10 microns.

### SUMMARY OF THE INVENTION

We have discovered that by controlling atmospheric conditions during the manufacture of a pharmaceutical composition of a drug capable of dissociating to produce a valproate ion, the problem of stickiness can be avoided even without the use of any special grade silicon dioxide and the pharmaceutical composition so prepared exhibits a low punch residue.

In one general aspect, there is provided an extended release pharmaceutical composition comprising a drug capable of dissociating to produce a valproate ion, and at least one extended release polymer; wherein the pharmaceutical composition is manufactured under controlled atmospheric conditions, for example at a temperature of from about 27° C. to about 35° C. and a relative humidity of less than about 40% and, more particularly, less than about 20%.

The extended release pharmaceutical composition provides the drug over a prolonged period of time in such a manner as to provide substantial level of plasma concentrations of the drug following once-a-day dosing.

In another general aspect, there is provided a process for the preparation of an extended release pharmaceutical composition of a drug capable of dissociating to produce a valproate ion. The process includes a) dry blending a mixture of a drug capable of dissociating to produce a valproate ion, and at least one extended release polymer; b) wet granulating the blend from step a); c) drying and sizing the wet granules; d) lubricating the granules of step c); and e) compressing into or filling into a suitable size solid dosage form; wherein the pharmaceutical composition is manufactured at a temperature of from about 27° C. to about 35° C. and a relative humidity of less than about 40% and, more particularly, less than about 20%.

In another general aspect, there is provided an extended release pharmaceutical composition of divalproex sodium comprising divalproex sodium, and at least one extended release polymer; wherein the pharmaceutical composition is manufactured at a temperature of from about 27° C. to about 35° C. and a relative humidity of less than about 40% and, more particularly, less than about 20%.

In another general aspect, there is provided a process for the preparation of an extended release pharmaceutical composition of divalproex sodium. The process includes a) dry blending a mixture of from about 10-90% divalproex sodium, and from about 7-65% of at least one extended release polymer; b) wet granulating the blend from step a); c) drying and sizing the wet granules and d) lubricating the granules from step c); e) compressing into or filling into suitable size solid dosage form; wherein all percentages are based upon the total weight of the pharmaceutical composition and the pharmaceutical composition is manufactured at a temperature of from about 27° C. to about 35° C. and a relative humidity of less than about 40% and, more particularly, less than about 20%.

In another general aspect, there is provided an extended release pharmaceutical composition of divalproex sodium. The composition includes a) from about 10-90% of divalproex sodium; b) from about 7-65% of hydroxypropyl methylcellulose; c) from about 0.5-18% of lactose and d) from about 0.5-5% of silicon dioxide; wherein all weight percentages are based upon the total weight of pharmaceutical composition and it is manufactured at a temperature of from about 27° C. to about 35° C. and a relative humidity of less than about 40% and, more particularly, less than about 20%.

In another general aspect, there is provided an extended release tablet dosage form comprising a drug capable of dissociating to produce a valproate ion, and at least one extended release polymer, wherein the tablet is manufactured at a temperature of from about 27° C. to about 35° C. and a relative humidity of less than about 40% and, more particularly, less than about 20% and provides a low punch residue as compared to the tablet prepared under normal conditions. Normal conditions under which the tablets are generally manufactured are temperature of about 22° C.-25° C. and a relative humidity 50% or more.

In another general aspect, there is provided an extended release tablet comprising a drug capable of dissociating to produce a valproate ion, and b) at least one extended release polymer, wherein the average residue on the tablet punch is less than about 0.3% w/w of the active ingredient.

In another general aspect, there is provided an extended release tablet composition of divalproex sodium. The composition comprising divalproex sodium, equivalent to about 100 mg to about 1100 mg of valproic acid and at least one extended release polymer, wherein the total tablet weight is less than about 1500 mg.

In another general aspect, there is provided an extended release once a day tablet of divalproex sodium comprising divalproex sodium, and at least one extended release polymer,

wherein said tablet exhibits the following dissolution profile, when measured in a type 2 dissolution apparatus, paddle, at 100 rpm, at a temperature of 37±0.5 C., in 500 ml of 0.1N HCl for 45 minutes, followed by 900 ml of 0.05M phosphate buffer containing 75 mM sodium lauryl sulfate, pH 5.5, for the remainder of the testing period:

a) no more than about 30% of total valproate is released after 3 hours of measurement in said apparatus;

b) from about 40 to about 70% of total valproate is released after 9 hours of measurement in said apparatus;

c) from about 50 to about 80% of total valproate is released after 12 hour of measurement in said apparatus, and;

d) not more than 85% of total valproate is released after 18 hours of measurement in said apparatus.

In another general aspect, there is provided an extended release once a day tablet of divalproex sodium comprising divalproex sodium and sufficient quantity of at least one extended release polymer, so that said tablet when ingested orally by healthy human subjects produces Cₘₐₓ (Maximum plasma concentration) and AUC_{0-∝} (Area under the plasma concentration vs. time curve from 0 hours to infinity) that is comparable to the Cₘₐₓ and AUC_{0-∝} value produced by the equivalent dose of Depakote® ER divalproex sodium extended release tablets.

In another general aspect, there is provided a method of treating mania, migraine and epilepsy using an extended release pharmaceutical composition comprising a drug capable of dissociating to produce a valproate ion, and at least one extended release polymer, wherein the pharmaceutical composition is manufactured under controlled atmospheric conditions.

### DETAILED DESCRIPTION OF THE INVENTION

The inventors have discovered two important characteristics in developing an extended release pharmaceutical composition of valproic acid and its derivatives, manufactured under controlled atmospheric conditions (temperature of from about 27° C. to about 35° C. and a relative humidity of less than about 40% and, more particularly, less than about 20%): (1) the formulation not only eliminates the problem of sticking but also imparts elegance to the composition, and (2) it also has reduced friability to an acceptable value. It was discovered that, it is not the use of microcrystalline cellulose or a special grade silicon dioxide, but the atmospheric conditions that are responsible for overcoming the problem of stickiness. Even the use of special grade silicon dioxide (as taught by U.S. Pat. No. 6,419,953) leads to sticking problems.

The term ‘about’ as used herein includes temperature and relative humidity conditions up to ±10% of the indicated values.

The term ‘pharmaceutical composition’ as used herein includes solid dosage forms such as tablet, capsule, pill, and the like. The tablets can be prepared by techniques known in the art and contain a therapeutically effective amount of the valproate compound and such excipients as are necessary to form the tablet by such techniques. Tablets and pills can additionally be prepared with enteric coatings and other release-controlling coatings for the purpose of acid protection, easy swallowing, etc.

The term ‘drug capable of dissociating to produce a valproate ion’ includes a compound which dissociates within the gastrointestinal tract, to produce a valproate ion including, but not limited to, valproic acid, the sodium salt of valproate, divalproex sodium, any of the various salts of valproic acid described below, and any of the prodrugs of valproic acid described below.

Valproic acid is known for its activity as an antiepileptic compound as described in the Physician Desk Reference, 55th Edition, page 422 (2001). Upon oral ingestion within the gastrointestinal tract, the acid moiety dissociates to form a carboxylate moiety (i.e. a valproate ion).

The sodium salt of valproic acid is also known in the art as an anti-epileptic agent. It is also known as sodium valproate and is described in The Merck Index, 12 Edition, page 1691 (1996).

Divalproex sodium is effective as an antiepileptic agent and is also used for migraine and bipolar disorders. It is a stable co-ordination compound comprising of sodium valproate and valproic acid in a 1:1 ratio and formed during the partial neutralization of valproic acid with 0.5 equivalent of sodium hydroxide. The amount of drug may vary from about 10% to about 90% by weight of the total pharmaceutical composition weight. Like valproic acid, it also dissociates within the gastrointestinal tract to form a valproate ion.

In addition to these specific compounds, one of ordinary skill in the art would readily recognize that the carboxylic moiety of the valproate compound might be functionalized in a variety of ways. This includes forming compounds that readily metabolize in-vivo to produce valproate, such as valproate amide (valpromide), as well as other pharmaceutically acceptable amides and esters of the acid (i.e. prodrugs). This also includes forming a variety of pharmaceutically acceptable salts.

Suitable pharmaceutically acceptable basic addition salts include, but are not limited to cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like.

Other possible compounds include pharmaceutically acceptable amides and esters. “Pharmaceutically acceptable ester” refers to those esters that retain, upon hydrolysis of the ester bond, the biological effectiveness and properties of the carboxylic acid and are not biologically or otherwise undesirable. The alcohol component of the ester will generally comprise (i) a C₂-C₁₂ aliphatic alcohol that can or can not contain one or more double bonds and can or can not contain branched carbons or (ii) a C₇-C₁₂ aromatic or heteroaromatic alcohols. This invention also contemplates the use of those compositions, which are both esters as described herein, and at the same time are the pharmaceutically acceptable salts thereof.

“Pharmaceutically acceptable amide” refers to those amides that retain, upon hydrolysis of the amide bond, the biological effectiveness and properties of the carboxylic acid and are not biologically or otherwise undesirable. This invention also contemplates the use of those compositions, which are both amides as described herein, and at the same time are the pharmaceutically acceptable salts thereof.

The term ‘extended release pharmaceutical composition’ as used herein includes any pharmaceutical composition that achieves the slow release of drug over an extended period of time, and includes both prolonged and controlled release compositions. This includes matrix systems, osmotic systems and membrane-controlled systems.

The extended release polymer may be a water-soluble polymer, or a water insoluble polymer (including waxes). Examples of water-soluble polymers include polyvinylpyrrolidone, hydroxypropylcellulose, hydroxypropylmethyl cellulose, methylcellulose, vinyl acetate copolymers, polysaccharides (such as alginate, xanthan gum, etc.), polyethylene oxide, methacrylic acid copolymers, maleic anhydride/methyl vinyl ether copolymers and derivatives and mixtures thereof. Examples of water-insoluble polymers include acrylates such as methacrylates, acrylic acid copolymers; cellulose derivatives such as ethylcellulose or cellulose acetate; polyethylene, and high molecular weight polyvinylalcohols. Examples of suitable waxes include fatty acids and glycerides.

The extended release pharmaceutical composition may be prepared by processes known in the prior art for example, by comminuting, mixing, granulation, melting, sizing, filling, drying, molding, immersing, coating, compressing etc.

In one general aspect, the extended release tablets may be prepared by wet granulation technique, comprising the steps of blending drug capable of dissociating to produce a valproate ion, extended release polymer and optionally pharmaceutically inert excipient; granulating with a granulating fluid or solution/dispersion of binder; drying and sizing the granules; optionally blending with pharmaceutically inert extragranular excipients; lubricating the granules/blend; compressing the lubricated blend into suitable sized tablets and; optionally coating with film forming polymer and coating additives.

In another general aspect, the extended release tablets may be prepared by dry granulation technique, comprising the steps of blending drug capable of dissociating to produce a valproate ion, extended release polymer and optionally pharmaceutically inert excipient; dry granulating the blend by roller compactor or slugging; lubricating the granules/blend; compressing the lubricated blend into suitable sized tablets and; optionally coating with film forming polymer and coating additives.

In another general aspect, the extended release tablets may be prepared by direct compression technique, comprising the steps of blending drug capable of dissociating as a valproate ion, extended release polymer and optionally pharmaceutically inert excipient; lubricating the blend; directly compressing the lubricated blend into suitable sized tablets and; optionally coating with film forming polymer and coating additives.

In another general aspect, the extended release tablets may be prepared by melt extrusion technique, comprising the steps of blending drug capable of dissociating as valproate ion, extended release polymer and optionally pharmaceutically inert excipient; melting the blend followed by solidifying into a compact mass; breaking the compact mass into granules; optionally blending with pharmaceutically inert extragranular excipients; lubricating the granules/blend; compressing the lubricated blend into suitable sized tablets and; optionally coating with film forming polymer and coating additives.

The term “pharmaceutically acceptable inert excipients” as used herein includes all excipients used in the art of manufacturing solid dosage forms. Common excipients include binders, diluents, surfactants, lubricants/glidants, coloring agents, and the like.

Examples of suitable binders include methyl cellulose, hydroxypropyl cellulose, hydroxypropyl methylcellulose, polyvinylpyrrolidone, gelatin, gum arabic, ethyl cellulose, polyvinyl alcohol, pullulan, pregelatinized starch, agar, tragacanth, sodium alginate, propylene glycol, and the like.

Suitable diluents include calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, sugar confectioners, and the like.

Surfactants include both non-ionic and ionic (cationic, anionic and zwitterionic) surfactants suitable for use in pharmaceutical dosage forms. These include polyethoxylated fatty acids and its derivatives, for example polyethylene glycol 400 distearate, polyethylene glycol-20 dioleate, polyethylene glycol 4-150 mono dilaurate, polyethylene glycol-20 glyceryl stearate; alcohol-oil transesterification products, for example polyethylene glycol-6 corn oil; polyglycerized fatty acids, for example polyglyceryl-6 pentaoleate; propylene glycol fatty acid esters, for example propylene glycol monocaprylate; mono and diglycerides for example glyceryl ricinoleate; sterol and sterol derivatives; sorbitan fatty acid esters and its derivatives, for example polyethylene glycol-20 sorbitan monooleate, sorbitan monolaurate; polyethylene glycol alkyl ether or phenols, for example polyethylene glycol-20 cetyl ether, polyethylene glycol-10-100 nonyl phenol; sugar esters, for example sucrose monopalmitate; polyoxyethylene-polyoxypropylene block copolymers known as “poloxamer”; ionic surfactants, for example sodium caproate, sodium glycocholate, soy lecithin, sodium stearyl fumarate, propylene glycol alginate, octyl sulfosuccinate disodium, palmitoyl camitine; and the like.

Examples of suitable lubricants/glidants include colloidal silicon dioxide, stearic acid, magnesium stearate, calcium stearate, talc, hydrogenated castor oil, sucrose esters of fatty acid, microcrystalline wax, yellow beeswax, white beeswax, and the like.

Coloring agents include any FDA approved colors for oral use.

The pharmaceutical composition may optionally be coated with functional and/or non-functional layers comprising film-forming polymers, if desired.

Examples of film-forming polymers include ethylcellulose, hydroxypropyl methylcellulose, hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit® RL and RS; and the like. Alternatively, commercially available coating compositions comprising film-forming polymers marketed under various trade names, such as Opadry® may also be used for coating.

The following examples are provided to enable one of ordinary skill in art to prepare dosage forms of the invention and should not be construed as limiting the scope of invention. In the following examples, the divalproex sodium tablets were prepared under controlled conditions (temperature from about 27° C. to about 35° C. and relative humidity less than about 20%), using the procedure as described below.

Divalproex sodium, lactose and hydroxypropyl methylcellulose were blended in a rapid mixer granulator. The granules were prepared adding the granulation fluid (purified water) to mixture of drug/polymer/lactose. The resulting granules were dried in a fluidized bed drier and sieved through suitable sieves. The dried granules were blended with talc and magnesium stearate and compressed into suitable sized tablets and coated with an aqueous dispersion of PEG 400 and Opadry.

### EXAMPLES 1-6

Examples 1-6

| | Wt/tablet (mg) | | | | | |
|---|---|---|---|---|---|---|
| Ingredient | 1 | 2 | 3 | 4 | 5 | 6 |
| Divalproex | 272.3 | 272.3 | 544.6 | 538.2 | 1076.4 | 1076.4 |
| sodium | | | | | | |
| Lactose | 10 | 10 | 125.4 | 131.8 | 10 | 25 |
| Hydroxypropyl | 300 | 350 | 270 | 300 | 245 | 275 |
| methylcellulose | | | | | | |
| Water | q.s. | q.s. | q.s. | q.s. | q.s. | q.s |
| Magnesium | 5 | 5 | 5 | 5 | 10 | 10 |
| Stearate | | | | | | |
| Talc | 7.7 | 7.7 | 15 | 15 | 28.6 | 28.6 |
| Colloidal Silicon | 5 | 5 | 10 | 10 | 10 | 10 |
| Dioxide | | | | | | |

### EXAMPLE 7

Tablets were also prepared as per the composition of Example 6 using the following procedure:

Divalproex sodium, hydroxypropyl methylcellulose and lactose were blended in a rapid mixer granulator. The granules were prepared adding the granulation fluid (dispersion of 0.5 mg/ml hydroxypropyl methylcellulose in purified water) to mixture of drug/polymer/lactose. The resulting granules were dried in a fluidized bed drier and sieved through suitable sieves. The dried granules were blended with talc and magnesium stearate and compressed into suitable sized tablets and coated with an aqueous dispersion of PEG 400 and Opadry.

The extended release tablets prepared according to Examples 1-6 were then evaluated for hardness and friability. Hardness of extended release tablets of divalproex sodium as per composition of Examples 1-6 was determined using Scheulinger Tablet hardness tester (for Examples 3-6) and Vankel Hardness tester (for Examples 1 & 2 ), the results of which are listed in Table 1.

TABLE 1
Hardness & friability of extended release tablets of divalproex sodium.

| | Exam- | Exam- | Exam- | Exam- | Exam- | Exam- |
|---|---|---|---|---|---|---|
| Ingredients | ple 1 | ple 2 | ple 3 | ple 4 | ple 5 | ple 6 |
| Hardness (kP) | 12-14 | 12-14 | 15-17 | 15-17 | 16-18 | 18-20 |
| Friability (% | 0.02 | 0.22 | 0.12 | 0.08 | 0.7 | 0.11 |
| Loss) | | | | | | |

The tablet of Example 4 and preferred tablet formulation B of U.S. Pat. No. 6,419,953, were prepared and evaluated for stickiness. These tablets were made on rotary press with punch of dimensions 19.2×9.3 mm and at a hardness of about 13-15 kP.

After 50 tablets, the tablet material was extracted from the punches using about 7.5 ml of acetonitrile and sonicated. The volume was then made up to 10 ml with water; this procedure was repeated for runs of 100, 150, 200, and 250 tablets. The extracts together with valproic acid calibration samples were measured by HPLC for content of valproic acid. The amount of valproic acid in the samples obtained from tablet formulation B was calculated from the standard curve and the total amount of valproic acid extracted from both the upper and lower punch was plotted against the amount of tablets made. An average value for stickiness was calculated from the slope of the regression line by forcing the y-intercept of the line through zero. The weight residue obtained from tablet formulation B of U.S. Pat. No. 6,419,953 with respect to valproic acid was 0.0189 mg/tablet.

On the other hand, a constant weight residue of 0.010 mg/tablet (0.1% w/w of active ingredient) was obtained from first 50 tablets of Example 4. Further, no increase in punch residue was observed irrespective of the number of tablets produced. The constant residue weight clearly indicates almost negligible sticking of composition to the punches, when manufacturing was done under conditions described herein.

Above data also indicates that divalproex sodium tablets when manufactured under controlled temperature and humidity conditions produce tablets with less friability.

Table 2 provides comparative dissolution data for the marketed Depakote® ER (500 mg) and the extended release tablets of divalproex sodium of Example 4. The testing was performed using type 2 USP dissolution apparatus, operating at 37° C. with a paddle rotating speed of 100 rpm. The tablets were tested in 500 ml of 0.1 N hydrochloric acid for first 45 min, followed by 900 ml of 0.05M phosphate buffer containing 75 mM sodium lauryl sulphate at pH 5.5.

TABLE 2
Comparative Dissolution profile of Divalproex sodium extended re-
lease tablets (equivalent to 500 mg valproic acid) of Example 4 and
Depakote ® (500 mg) ER tablets

Cumulative percentage (%)
release of valproic acid

| | | Depakote ® ER tablet (500 |
|---|---|---|
| Time (h) | Example 4 | mg) |
| 1 | 9 | 8 |
| 3 | 22 | 19 |
| 5 | 33 | 29 |
| 9 | 49 | 44 |
| 12 | 59 | 60 |
| 18 | 79 | 102 |

Table 3 provides comparative dissolution data for the marketed Depakote® ER (2×500 mg) and the extended release tablets of divalproex sodium of Examples 5-6. The testing was performed using type 2 USP dissolution apparatus with a paddle speed of 100 rpm. The tablets were tested in 900 ml phosphate buffer (pH 6.8) with 1% sodium lauryl sulphate. The tablets were kept in sinker basket of 10# and the height of paddle was 4.5 cm from the bottom.

TABLE 3
Comparative Dissolution profile of Divalproex sodium extended re-
lease tablets (equivalent to 1000 mg valproic acid) of Example 5-6
and Depakote ® (2 × 500 mg) ER tablets

| | Cumulative percentage (%) release of valproic acid | | |
|---|---|---|---|
| | | | Depakote ® ER (2 × |
| Time (h) | Example 5 | Example 6 | 500 mg) |
| 1 | 16 | 15 | 17 |
| 2 | 26 | 22 | 24 |
| 4 | 39 | 34 | 35 |
| 8 | 59 | 53 | 50 |
| 12 | 75 | 64 | 61 |
| 16 | 88 | 75 | 69 |
| 20 | 98 | 94 | 83 |
| 24 | 105 | 98 | 96 |

Further, bioavailability study of the divalproex sodium extended release tablet (500 mg) of Example 4 was carried out on healthy male volunteers (n=12) taking Depakote® ER tablet (500 mg) as the reference, the results of which are represented in Table 4.

Open randomized, 2 treatment, 2 period, 2 sequence, single dose crossover, was used for comparative bioavailability study of divalproex sodium 500 mg extended release tablet against Depakote® ER tablets—500 mg of Abbott laboratories under fed conditions:

TABLE 4
Pharmacokinetic parameters obtained through the bioavailability
studies of divalproex sodium extended release tablets and Depakote ®
ER tablets (500 mg).

| Pharmacokinetic | Cₘₐₓ* | AUC₀₋ₜ** | AUC_{0-oc}*** | Tₘₐₓ**** |
|---|---|---|---|---|
| parameter | (ng/ml) | (ng · h/ml) | (ng · h/ml) | (h) |
| Divalproex sodium | 50.7 | 1592.31 | 1811.54 | 18.67 |
| extended release | | | | |
| tablet of Example 4 | | | | |
| (Test) | | | | |
| Depakote ® ER 500 | 47.75 | 1599.74 | 1940.07 | 20 |
| mg tablet (Ref.) | | | | |
| Test/Ref. (90% | 106.91 | 99.21 | 92.21 | |
| confidence | (99.63- | (87.41- | (81.76- | — |
| interval) | 114.72) | 112.61) | 104) | |

*Cₘₐₓ = Maximum plasma concentration, **AUC₀₋ₜ = Area under the plasma concentration vs time curve from 0 hrs to the time of last sample collected, ***AUC_{0-oc} = Area under the plasma concentration vs. time curve from 0 hrs to infinity, ****Tₘₐₓ Time to attain maximum plasma concentration

AUC_{0-∝} for Divalproex sodium was within 80-125% as per FDA guidelines on bioequivalence (Table 4). Above results show that divalproex sodium 500 mg extended release tablets prepared as per Example 4 have bioavailability comparable to the reference product, Depakote® ER tablet 500 mg.

The extended release tablet formulations of the present invention thus provide an effective delivery system for the once daily administration of valproic acid (divalproex sodium) to patients in need of such treatment.

Bioavailability study of the Divalproex sodium (1000 mg) ER tablet of example 6 was carried out on healthy male volunteers (n=11) taking Depakote® ER tablet (2×500 mg) as the reference, the results of which are represented in Table 5. The objective of this study was to show that a formulation of example 6 provides an activity and safety profile that is similar or better to one obtained with an equivalent product in the market.

Open randomized, 2 treatment, 2 period, 2 sequence, single dose crossover, comparative bioavailability study of Divalproex sodium extended release tablets against 2×500 mg ER tablets of Depakote® ER tablets was performed under fed conditions. Comparative pharmacokinetic parameters thus obtained are listed in Table 5.

TABLE 5
Comparative pharmacokinetic parameters for tablets of Divalproex
Sodium ER and Depakote ® ER tablets (500 mg × 2).

| Pharmacokinetic | Cₘₐₓ* | AUC₀₋ₜ** | AUC_{0-oc}*** | Tₘₐₓ**** |
|---|---|---|---|---|
| parameter | (ng/ml) | (ng · h/ml) | (ng · h/ml) | (h) |
| Divalproex | 70.31 | 1827.86 | 1981.31 | 9.91 |
| sodium ER tablet | | | | |
| of example 6 | | | | |
| (Test) | | | | |
| Depakote ® ER | 63.61 | 1899.77 | 2099.67 | 14.36 |
| (500 mg × 2) | | | | |
| tablet (Ref.) | | | | |
| Test/Ref. (90% | 110.02 | 95.41 | 93.81 | — |
| confidence | (99.61- | (86.08-105.76) | (83.99-104.78) | |
| interval) | 121.52) | | | |

*Cₘₐₓ = Maximum plasma concentration, **AUC₀₋ₜ = Area under the plasma concentration vs time curve from 0 hours to the time of last sample collected, ***AUC_{0-oc} = Area under the plasma concentration vs. time curve from 0 hours to infinity, and ****Tₘₐₓ = Time to attain maximum plasma concentration

AUC_{0-∝} for Divalproex sodium was within 80-125% as shown in Table 3. The results show that Divalproex Sodium 1000 mg extended release tablets prepared as per the examples described herein have bioavailability comparable to the reference product, Depakote® ER tablet (500×2 mg).

While several particular forms of the invention have been illustrated and described, it will be apparent that various modifications and combinations of the invention detailed in the text can be made without departing from the spirit and scope of the invention. Further, it is contemplated that any single feature or any combination of optional features of the inventive variations described herein may be specifically excluded from the claimed invention and be so described as a negative limitation. Accordingly, it is not intended that the invention be limited, except as by the appended claims.

We claim:

1. An extended release pharmaceutical composition comprising:
a) a drug capable of dissociating to produce a valproate ion; and
b) at least one extended release polymer;
wherein the pharmaceutical composition is manufactured under controlled atmospheric conditions.

2. The extended release pharmaceutical composition according to claim 1, wherein the drug is selected from the group consisting of valproic acid, a pharmaceutically acceptable salt or ester of valproic acid, divalproex sodium, and valpromide.

3. The extended release pharmaceutical composition according to claim 2, wherein the drug is divalproex sodium.

4. The extended release pharmaceutical composition according to claim 1, wherein the controlled atmospheric conditions comprise controlling relative humidity.

5. The extended release pharmaceutical composition according to claim 4, wherein the relative humidity is less than about 40%.

6. The extended release pharmaceutical composition according to claim 5, wherein the relative humidity is less than about 20%.

7. The extended release pharmaceutical composition according to claim 1, wherein the controlled atmospheric conditions comprise controlling temperature.

8. The extended release pharmaceutical composition according to claim 7, wherein the temperature is from about 27° C. to about 35° C.

9. The extended release pharmaceutical composition according to claim 1, wherein the extended release polymer is a water-soluble polymer or a water insoluble polymer.

10. The extended release pharmaceutical composition according to claim 9, wherein the water-soluble polymer is selected from the group consisting of polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives and mixtures thereof.

11. The extended release pharmaceutical composition according to claim 9, wherein the water-insoluble polymer is selected from the group consisting of methacrylates, acrylic acid copolymers, ethylcellulose, cellulose acetate, polyethylene, and high molecular weight polyvinylalcohols.

12. The extended release pharmaceutical composition according to claim 1, wherein the pharmaceutical composition is a tablet, capsule, or a pill.

13. The extended release pharmaceutical composition according to claim 12, wherein the pharmaceutical composition is a tablet.

14. An extended release tablet comprising a) a drug capable of dissociating to produce a valproate ion, and b) at least one extended release polymer, wherein the tablet exhibits a low punch residue when manufactured under controlled atmospheric conditions as compared to the tablet prepared under normal conditions.

15. The extended release pharmaceutical composition according to claim 14, wherein the controlled atmospheric conditions comprise controlling relative humidity.

16. The extended release pharmaceutical composition according to claim 15, wherein the relative humidity is less than about 40%.

17. The extended release pharmaceutical composition according to claim 16, wherein the relative humidity is less than about 20%.

18. The extended release pharmaceutical composition according to claim 14, wherein the controlled atmospheric conditions comprise controlling temperature.

19. The extended release pharmaceutical composition according to claim 18, wherein the temperature is from about 27° C. to about 35° C.

20. An extended release tablet comprising a drug capable of dissociating to produce a valproate ion, and b) at least one extended release polymer, wherein the average residue on the tablet punch is less than about 0.3% w/w of the active ingredient.

21. The extended release tablet according to claim 14 or 20, wherein the drug capable of dissociating as a valproate ion is selected from the group consisting of valproic acid, a pharmaceutically acceptable salt or ester of valproic acid, divalproex sodium, and valpromide.

22. The extended release pharmaceutical composition according to claim 21, wherein the drug is divalproex sodium.

23. The extended release pharmaceutical composition according to claim 14 or 20, wherein the extended release polymer is a water-soluble polymer or a water insoluble polymer.

24. The extended release pharmaceutical composition according to claim 23, wherein the water-soluble polymer is selected from the group consisting of polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives and mixtures thereof.

25. The extended release pharmaceutical composition according to claim 23, wherein the water-insoluble polymer is selected from the group consisting of methacrylates, acrylic acid copolymers, ethylcellulose, cellulose acetate, polyethylene, and high molecular weight polyvinylalcohols.

26. An extended release pharmaceutical composition comprising
a) divalproex sodium, and
b) at least one extended release polymer;
wherein the pharmaceutical composition is manufactured at a temperature of about 27° C. to about 35° C. and relative humidity of less than about 20%.

27. The extended release pharmaceutical composition according to claim 26, wherein the divalproex sodium is present in an amount from about 10% to about 90% by weight of the total pharmaceutical composition weight.

28. The extended release pharmaceutical composition according to claim 26, wherein the extended release polymer is a water-soluble polymer or a water insoluble polymer.

29. The extended release pharmaceutical composition according to claim 28, wherein the water-soluble polymer is selected from the group consisting of polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives and mixtures thereof.

30. The extended release pharmaceutical composition according to claim 29, wherein the water-soluble polymer is hydroxypropyl methylcellulose.

31. The extended release pharmaceutical composition according to claim 30, wherein the hydroxypropyl methylcellulose is present in an amount from about 7% to about 65% by weight of the total pharmaceutical composition weight.

32. The extended release pharmaceutical composition according to claim 28, wherein the water-insoluble polymer is selected from the group consisting of methacrylates, acrylic acid copolymers, ethylcellulose, cellulose acetate, polyethylene, and high molecular weight polyvinylalcohols.

33. The extended release pharmaceutical composition according to claim 26, wherein the pharmaceutical composition is a tablet, capsule, or a pill.

34. The extended release pharmaceutical composition according to claim 33, wherein the pharmaceutical composition is a tablet.

35. The extended release pharmaceutical composition according to claim 1, 14, 20 or 26, wherein the extended release pharmaceutical composition further comprising one or more pharmaceutically inert excipients.

36. The extended release pharmaceutical composition according to claim 35 wherein one or more pharmaceutically inert excipients comprise one or more glidants, lubricants, diluents, binders, colorants, and flavoring agents.

37. An extended release pharmaceutical composition comprising:
a) from about 10-90% of divalproex sodium,
b) from about 7-65% of hydroxypropyl methylcellulose,
c) from about 0.5-18% of lactose, and
d) from about 0.5-5% colloidal silicon dioxide;
wherein all percentages are based upon the total weight of the pharmaceutical composition and it is manufactured at a temperature of from about 27° C. and about 35° C. and relative humidity of less than about 20%.

38. An extended release tablet composition comprising divalproex sodium, equivalent to about 100 mg to about 1100 mg of valproic acid and at least one extended release polymer, wherein the total tablet weight is less than about 1500 mg.

39. The extended release tablet composition according to claim 38, wherein composition comprises divalproex sodium equivalent to 1000 mg of valproic acid.

40. The extended release tablet composition according to claim 39, wherein the extended release polymer is less than 20% by weight of total tablet weight.

41. The extended release tablet composition according to claim 38, wherein the extended release polymer is a water-soluble polymer or a water insoluble polymer.

42. The extended release tablet composition according to claim 41, wherein the water-soluble polymer is selected from the group consisting of polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives and mixtures thereof.

43. The extended release tablet composition according to claim 41, wherein the water-insoluble polymer is selected from the group consisting of methacrylates wherein the water-insoluble polymer, acrylic acid copolymers, ethylcellulose, cellulose acetate, polyethylene, and high molecular weight polyvinylalcohols.

44. The extended release tablet composition according to claim 38, which is suitable for once-a-day dosing.

45. A process for the preparation of an extended release pharmaceutical composition, the process comprising:
a) blending a drug capable of dissociating to produce a valproate ion, and at least one extended release polymer,
b) optionally granulating the blend,
c) lubricating the blend of step a) or granules of step b), and
d) compressing into or filling into a suitable size solid dosage form;
wherein the pharmaceutical composition is manufactured under controlled atmospheric conditions.

46. A process for the preparation of an extended release pharmaceutical composition, the process comprising:
a) blending divalproex sodium, and at least one extended release polymer,
b) optionally granulating the blend,
c) lubricating the blend of step a) or granules of step b), and
d) compressing into or filling into a suitable size solid dosage form;
wherein the pharmaceutical composition is manufactured under controlled atmospheric conditions.

47. The process according to claim 45 or 46, wherein the granulating comprises one of a wet granulation, dry granulation, or a melt extrusion technique.

48. The process according to claim 47, wherein the granulation is carried out by a wet granulation technique.

49. A process for the preparation of an extended release pharmaceutical composition of divalproex sodium, the process comprising:
a) dry blending a mixture of from about 10-90% divalproex sodium, and from about 7-65% of at least one extended release polymer;
b) wet granulating the blend from step a);
c) drying and sizing the wet granules;
d) lubricating the granules from step c); and
e) compressing into or filling into a suitable size solid dosage form;
wherein all percentages are based upon the total weight of the pharmaceutical composition and it is manufactured under controlled atmospheric conditions.

50. The process according to claim 49, wherein in step e) granules are compressed into solid dosage form.

51. The process according to claim 50, wherein the solid dosage form is a tablet.

52. The process according to claim 49, wherein in step e) granules are filled into a suitable size solid dosage form.

53. The process according to claim 52, wherein the solid dosage form is a capsule.

54. The extended release pharmaceutical composition according to claim 45, 46 or 49, wherein the controlled atmospheric conditions comprise controlling relative humidity.

55. The extended release pharmaceutical composition according to claim 54, wherein the relative humidity is less than about 40%.

56. The extended release pharmaceutical composition according to claim 55, wherein the relative humidity is less than about 20%.

57. The extended release pharmaceutical composition according to claim 45, 46, or 49, wherein the controlled atmospheric conditions comprise controlling temperature.

58. The extended release pharmaceutical composition according to claim 57, wherein the temperature is from about 27° C. to about 35° C.

59. An extended release tablet comprising:
a) divalproex sodium, and
b) at least one extended release polymer;
wherein said tablet when measured in a type 2 dissolution apparatus, paddle, at 100 rpm, at a temperature of 37±0.5 C., in 500 ml of 0.1N HCl for 45 minutes, followed by 900 ml of 0.05M phosphate buffer containing 75 mM sodium lauryl sulfate, pH 5.5, for the remainder of the testing period exhibits an in vitro dissolution profile as follows:
i. no more than about 30% of total valproate is released after 3 hours of measurement in said apparatus;
ii. from about 40 to about 70% of total valproate is released after 9 hours of measurement in said apparatus;
iii. from about 50 to about 80% of total valproate is released after 12 hour of measurement in said apparatus, and;
iv. not more than 85% of total valproate is released after 18 hours of measurement in said apparatus.

60. The extended release tablet according to claim 59, wherein the tablet is manufactured at a temperature of about 27° C. to about 35° C. and a relative humidity of less than about 20%.

61. The extended release tablet according to claim 59, wherein said tablet exhibits the following in vitro dissolution profile:
a. from about 15% to about 30% of total valproate is released after 3 hours of measurement in said apparatus;
b. from about 40% to about 70% of total valproate is released after 9 hours of measurement in said apparatus;
c. from about 50% to about 80% of total valproate is released after 12 hours of measurement in said apparatus, and;
d. not more than 85% of total valproate is released after 18 hours of measurement in said apparatus.

62. The extended release pharmaceutical composition according to claim 59, wherein the extended release pharmaceutical composition further comprising one or more pharmaceutically inert excipients.

63. The extended release tablet according to claim 37 or 59, which when ingested orally by healthy human subjects produces a Cₘₐₓ and AUC_{0-∝} which is comparable to the Cₘₐₓ and AUC_{0-∝} values generated by equivalent dose of Depakote® divalproex sodium extended release tablet.

64. The extended release tablet according to claim 59 which is suitable for once-a-day dosing.

65. A method of treating epilepsy, migraine and bipolar disorders by administering an extended release pharmaceutical composition comprising:
a) a drug capable of dissociating to produce a valproate ion, and
b) at least one extended release polymer;
wherein the pharmaceutical composition is manufactured at a temperature of from about 27° C. to about 35° C. and relative humidity of less than about 20%.

66. The extended release pharmaceutical composition according to claim 65, wherein the drug capable of dissociating to produce a valproate ion is selected from the group consisting of valproic acid, a pharmaceutically acceptable salt or ester of valproic acid, divalproex sodium, and valpromide.

67. The extended release pharmaceutical composition according to claim 66, wherein the drug is divalproex sodium.

68. A method of treating epilepsy, migraine and bipolar disorders by administering an extended release pharmaceutical composition comprising:
a) divalproex sodium, and
b) at least one extended release polymer;
wherein the pharmaceutical composition is manufactured at a temperature of from about 27° C. to about 35° C. and relative humidity of less than about 20%.

69. The extended release pharmaceutical composition according to claim 65 or 68, wherein the extended release polymer is a water-soluble polymer or a water insoluble polymer.

70. The extended release pharmaceutical composition according to claim 69, wherein the water-soluble polymer is selected from the group consisting of polyvinylpyrrolidone, hydroxypropylcellulose, hydroxypropyl methylcellulose, methylcellulose, vinyl acetate copolymers, sodium alginate, xanthan gum, polyethylene oxide, methacrylic acid copolymers, maleic anhydride/methyl vinyl ether copolymers and derivatives and mixtures thereof.

71. The extended release pharmaceutical composition according to claim 69, wherein the water-insoluble polymer is selected from the group consisting of methacrylates, acrylic acid copolymers, ethylcellulose, cellulose acetate, polyethylene, and high molecular weight polyvinylalcohols.
